(19) United States
(12) Patent Application Publication
(10) Pub. No.: US 2008/0213363 A1
(43) Pub. Date: Sep. 4, 2008

(54) Methods and compositions for delivering 5-HT3 antagonists across the oral mucosa

(76) Inventors: Nikhilesh N. Singh, Mill Valley, CA (US); Natasha N. Singh, Mill Valley, CA (US)

Correspondence Address: O''Melveny & Myers LLP;IP&T Calendar Department LA-1118, 400 South Hope Street, Los Angeles, CA, 90071-2899, US

(21) Appl. No.: 10/763,641

(22) Filed: Jan. 22, 2004

Related U.S. Application Data
(60) Provisional application No. 60/442,475, filed on Jan. 23, 2003.

(51) Int. Cl. A61K 9/22; A61K 9/68; A61K 31/4164; A61K 31/47; A61K 31/439; A61K 31/437; A61P 1/08

(52) U.S. Cl. 424/468; 424/48; 514/397; 514/307; 514/413; 514/292; 514/294

(57) ABSTRACT

Compositions, methods, and formulations for delivering 5-HT₃ antagonists across the oral mucosa are described. In some variations, the described compositions comprise at least one 5-HT₃ antagonist, which is at least partly in an ionized form, and a buffer system. The buffer system generally comprises at least two different buffering agents and is capable of changing the pH of saliva from an arbitrary initial pH to a predetermined final pH, and of sustaining the final pH for a period of time. The predetermined final pH favors substantially complete conversion of the ionized form to the un-ionized form. In other variations, the described compositions comprise a 5-HT₃ antagonist, at least partly in an un-ionized form, and a buffer system. In these variations, the buffer system is capable of providing an adjusted salival pH such that the 5-HT₃ antagonist remains in its un-ionized form. Dissolving tablets are also described.

### CROSS REFERENCE TO RELATED APPLICATIONS

This application claims priority to U.S. provisional patent application Ser. No. 60/442,475 entitled “Therapeutic Agent Delivery Compositions for Buccal Cavity Absorption of Antagonists of 5HT₃ Receptors,” filed on Jan. 23, 2003, which is hereby incorporated by reference in its entirety.

### TECHNICAL FIELD

The present invention relates generally to the field of drug delivery across the oral mucosa, and more specifically, to the delivery of 5-HT₃ antagonists across the oral mucosa.

### BACKGROUND

The 5-HT₃ antagonists have become the therapeutic agents of choice for the management and prevention of chemotherapy and post-operative induced nausea and vomiting in patients. These agents are thought to act by blocking serotonin receptors in the enterochromaffin cells in the gastrointestinal tract, and in the area postrema of the brain. Most typically, the 5-HT₃ antagonists are administered intravenously for immediate intervention, or orally for maintenance therapy.

When delivered orally, the 5-HT₃ antagonists are typically formulated as tablets, capsules, or liquids, which are swallowed. Oral administration, however, has several disadvantages, such as drug losses during hepatic first pass metabolism, during enzymatic degradation within the GI tract, and during absorption. These drug losses not only increase the variability in drug response, but also often require that the medicament be given in greater initial doses. In addition, because the drug has to pass through the gastrointestinal system in order to enter the blood stream, the time to reach a therapeutic effect may be quite long, typically around forty-five minutes or longer.

Accordingly, other routes of drug administration have been investigated, including those involving transport across the mucous membranes. Of the various mucous membranes (e.g., oral, rectal, vaginal, ocular, nasal, etc.), drug delivery across the oral mucosa seems to be the most easily tolerated by patients. In addition to avoiding the problems with traditional oral administration, drug delivery across the oral mucosa has certain other advantages, due to the properties of the oral mucosa itself. For example, the mucous membranes of the oral cavity are highly vascularized and well supplied with lymphatic drainage sites.

In general, the mucous membranes of the oral cavity can be divided into five main regions: the floor of the mouth (sublingual), the cheeks (buccal), the gums (gingival), the roof of the mouth (palatal), and the lining of the lips. These regions differ from each other with respect to their anatomy, drug permeability, and physiological response to drugs. For example, in terms of permeability, sublingual is more permeable than buccal, which is more permeable than palatal. This permeability is generally based on the relative thickness and degree of keratinization of these membranes, with the sublingual mucosa being relatively thin and non-keratinized, the buccal mucosa being thicker and non-keratinized, and the palatal mucosa being intermediate in thickness, but keratinized.

In addition to the differences in permeability of the various mucous membranes, the extent of drug delivery is also affected by the properties of the drug to be delivered. The ability of a molecule to pass through any mucous membrane is dependent upon its size, its lipid solubility, and the extent to which it is ionized, among other factors.

The extent to which a drug is ionized has further been investigated with respect to drug delivery. Ionization is dependent on the dissociation constant, or pKa of the molecule, and the pH of the molecule's surrounding environment. In its un-ionized form, a drug is sufficiently lipophilic to traverse a membrane via passive diffusion. In fact, according to the pH partition hypothesis, only un-ionized, non-polar drugs will penetrate a lipid membrane.

At equilibrium, the concentrations of the un-ionized form of the drug are equal on both sides of the membrane. Therefore, as the percentage of un-ionized form of a drug is increased, transmucosal absorption of the drug is correspondingly increased. Maximum absorption across the membrane is thought to occur when a drug is 100% in its un-ionized form. Similarly, absorption across the membrane decreases as the extent of ionization increases. Therefore, one may influence the extent of drug absorption across the mucous membranes of the oral cavity by altering the pH of salival environment.

Some of the known transmucosal dosage forms include the use of a single buffering agent in order to change the pH of the saliva. However, these single buffering agents typically react with an acid or a base to create a final pH that is dependent upon the initial pH of the saliva of the user. A buffering agent used to attain a final pH that is dependent upon the initial pH of the user results in great variability. The extent of ionization, and hence the extent of absorption across the mucous membranes cannot be predicted with any sort of accuracy. This may pose significant problems when trying to calculate precise dosages. In addition, a single buffering agent is typically not capable of sustaining a given pH over a period of time. While other investigators have disclosed the use of more than one buffering agent, these aforementioned problems are not easily cured by the nonchalant addition of an extra buffering agent. That is, a buffering system capable of achieving and sustaining a final pH, independent of the initial pH in order to increase transmucosal absorption, has not heretofore been demonstrated.

Similarly, a buffer system that facilitates substantially complete conversion of the ionized to the un-ionized form in a short period of time so as to cause rapid delivery of practically an entire drug dose across the oral mucosa has not heretofore been demonstrated. Previous dosage forms resulted in great variability in drug delivery, due to the variability in the rates at which a drug was released from its carrier. That is, the rates of drug release in previously described chewing gums or lozenges are largely dependent upon the rate of chewing or sucking of the user. The variability in these rates from user to user further exacerbates the ability to predict the final amount of drug that will enter systemic circulation. In addition, the rate of drug release from the carrier is further dependent upon the ability of the drug to be released therefrom. Often times, the carrier (e.g., gum base) strongly adheres to the drug, making at least portions of the drug unavailable for absorption.

Accordingly, compositions for delivering therapeutic agents, and more specifically, 5-HT₃ antagonists, across the oral mucosa would be desirable. Similarly, compositions for delivering 5-HT₃ antagonists across the oral mucosa having a buffer system that produces a final pH, independent of the initial pH, and sustains that final pH for a given period of time would be desirable. In addition, compositions capable of rapidly facilitating substantially complete conversion of a 5-HT₃ antagonist from its ionized to its un-ionized form, or capable of maintaining a 5-HT₃ antagonist in its un-ionized form if initially present as such, would also be desirable. In addition, a wide variety of dosage forms for delivery across the oral mucosa would be desirable.

### SUMMARY

Described herein are compositions and methods for delivering at least one 5-HT₃ antagonist across the oral mucosa. In general, the compositions comprise at least one 5-HT₃ antagonist, and a buffer system. In some variations, the 5-HT₃ antagonist is at least partly in an ionized form and the ionized form is capable of being converted into an un-ionized form. In these variations, the buffer system comprises at least two different buffering agents and is capable of changing the pH of saliva from an arbitrary initial pH to a predetermined final pH, independent of the arbitrary initial pH, and of sustaining the predetermined final pH for a period of time. The predetermined final pH favors substantially complete conversion of the ionized form to the un-ionized form.

In some variations, the predetermined final pH is within a range of from about 7.1 to about 11.5, or within a range of from about 9 to about 11. The compositions may be formulated as lozenges, chewing gums, or dissolving tablets. In some variations, the compositions are formulated as lozenges or as dissolving tablets. In other variations, the compositions are formulated as chewing gums and further comprise a gum base. The gum base typically comprises at least one hydrophobic polymer and at least one hydrophilic polymer. In some variations, the at least one hydrophilic polymer and the at least one hydrophobic polymer are independently selected from the group consisting of a natural polymer, a synthetic polymer, and mixtures thereof, for example, selected from the group consisting of a butadiene-styrene copolymer, butyl rubber, polyethylene, polyisobutylene, polyvinyl acetate phthalate, and mixtures thereof.

In some variations, the predetermined final pH favors at least 80%, at least 95%, or at least 99% conversion of the ionized form to the un-ionized form. In some variations, this conversion occurs in 10 minutes or less. In other variations the predetermined final pH is sustained for a period of at least 5 minutes, at least 10 minutes, or at least 20 minutes.

The buffering agents may be selected from the group consisting of a mixture of a weak acid and a salt of the weak acid, and a mixture of a first base and a second base, the second base being weaker than the first base. For example, the mixture of the first base and second base may be selected from the group consisting of sodium carbonate and sodium bicarbonate, potassium carbonate and potassium bicarbonate, and magnesium carbonate and magnesium bicarbonate. Similarly, the mixture of the weak acid and the salt of the weak acid may be acetic acid and sodium acetate. In some variations, one buffering agent is sodium bicarbonate and one buffering agent is sodium carbonate. In other variations, one buffering agent is potassium bicarbonate and one buffering agent is potassium carbonate.

The buffering agents may be present in a wide range of compositional ratios. For example, the buffering agents may be in a weight ratio of from about 2:1 to 1:2, from about 3:1 to 1:3, from about 5:1 to 1:5, or from about 10:1 to 1:10. In some variations, the buffering agents are in a 1:1 ratio by weight. The compositions may further comprise a penetration enhancer.

The 5-HT₃ antagonist may be selected from the group consisting of ondansetron, palonosetron, tropisetron, lerisetron, alosetron, granisetron, dolasetron, bernesetron, ramosetron, azaseteron, itasetron, zacopride, and cilasentron. In some variations, the 5-HT₃ antagonist is ondansetron. In some variations, the 5-HT₃ antagonist is ondansetron and the composition provides a maximum plasma concentration/time to achieve maximum plasma concentration (Cₘₐₓ/Tₘₐₓ) within a range of about 0.15 ng/ml×min to about 0.6 ng/ml×min. In other variations, the 5-HT₃ antagonist is ondansetron and the composition provides a Cₘₐₓ/Tₘₐₓ within a range of about 0.4 ng/ml×min to about 0.6 ng/ml×min. Similarly, in some variations, the 5-HT₃ antagonist is ondansetron and the membrane permeability of ondansetron is in the range of about 0.3 cm/s to about 3.25 cm/s.

Dissolving tablets for delivering a 5-HT₃ antagonist across the oral mucosa are also described. Typically, these dissolving tablets comprise a 5-HT₃ antagonist, a protecting agent, and a buffer system. In these variations, the 5-HT₃ antagonist is at least partly in an ionized form and the ionized form is capable of being converted into an un-ionized form. The protecting agent coats at least a portion of the 5-HT₃ antagonist. The buffer system typically comprises at least two different buffering agents and is capable of changing the pH of saliva from an arbitrary initial pH to a predetermined final pH, independent of the arbitrary initial pH, and of sustaining the predetermined final pH for a period of time. The predetermined final pH favors substantially complete conversion of the ionized form to the un-ionized form. The dissolving tablet may further comprise a compound selected from the group consisting of a binder, a filler, a flavoring agent, a scenting agent, a coloring agent, a preservative, a plasticizer, a penetration enhancer, an elastomeric solvent, and mixtures thereof.

The 5-HT₃ antagonist of the dissolving tablet is typically selected from the group consisting of ondansetron, palonosetron, tropisetron, lerisetron, alosetron, granisetron, dolasetron, bernesetron, ramosetron, azaseteron, itasetron, zacopride, and cilasentron. In some variations, the 5-HT₃ antagonist is ondansetron.

Also described herein are compositions for delivering a 5-HT₃ antagonist across the oral mucosa when the 5-HT₃ antagonist is initially, at least partly in an un-ionized form and the un-ionized form is capable of being converted into an ionized form at the normal salival pH. These compositions typically comprise at least one 5-HT₃ antagonist and a buffer system. The buffer system typically comprises at least one buffering agent, and is capable of providing an adjusted salival pH such that the 5-HT₃ antagonist remains in its un-ionized form.

In some variations, the buffer system is capable of maintaining the adjusted salival pH for a predetermined period of time, for example, within the range of 5 to 10 minutes. The compositions may be formulated as a lozenge, a chewing gum, or a dissolving tablet.

Methods for treating nausea are also described. In general, the methods comprise the step of delivering a therapeutically effective amount of a 5-HT₃ antagonist across the oral mucosa. In some variations, the step of delivering the 5-HT₃ antagonist comprises the step of providing a composition comprising a 5-HT₃ antagonist and a buffer system. In these variations, the 5-HT₃ antagonist is at least partly in an un-ionized form and the un-ionized form is capable of being converted into an ionized form at the normal salival pH. The buffer system comprises at least one buffering agent and is capable of providing an adjusted salival pH such that the 5-HT₃ antagonist remains in its un-ionized form.

In other variations, the step of delivering the 5-HT₃ antagonist across the oral mucosa comprises the step of providing a composition comprising a 5-HT₃ antagonist, a protecting agent, and a buffer system. In these variations, the 5-HT₃ antagonist is at least partly in an ionized form and the ionized form is capable of being converted into an un-ionized form. The protecting agent coats at least a portion of the 5-HT₃ antagonist, and the buffer system comprises at least two different buffering agents and is capable of changing the pH of saliva from an arbitrary initial pH to a predetermined final pH, independent of the arbitrary initial pH, and of sustaining the predetermined final pH for a period of time. The predetermined final pH favors substantially complete conversion of the ionized form to the un-ionized form.

### BRIEF DESCRIPTION OF THE DRAWING

FIG. 1 is a graph comparing the mean ondansetron concentration in the blood versus time for an illustrative 8 mg composition, as described herein, with an 8 mg Zofran® tablet.

### DETAILED DESCRIPTION

Described herein are compositions for delivering at least one 5-HT₃ antagonist across the oral mucosa. In general, the compositions comprise at least one 5-HT₃ antagonist and a buffer system. As described in more detail below, a variety of different 5-HT₃ antagonists may be selected for delivery across the oral mucosa. Similarly, the compositions may be formulated to provide a variety of different dosage forms. For example, the compositions may be formulated as chewing gums, lozenges, or dissolving tablets.

The compositions described herein may also include additional compounds, such as a binder, a filler, a flavoring agent, a scenting agent, a coloring agent, a preservative, a softening agent, a penetration enhancer, an elastomeric solvent, or mixtures thereof. The penetration enhancers may be of the type that alters the nature of the oral mucosa to enhance penetration, or of the type that alters the nature of the 5-HT₃ antagonist to enhance penetration through the oral mucosa. Suitable penetration enhancers that may be used with the compositions and methods described herein include polyoxyethylene 23-lauryl ether, aprotin, azone, benzalkonium chloride, cetylpyridinium chloride, cetyltrimethylammonium bromide, cyclodextrin, dextran sulfate, lauric acid, propylene glycol, lysophosphatidylcholine, menthol, methoxysalicylate, methyloleate, oleic acid, phosphatidylcholine, polyoxyethylene, polysorbate 80, sodium ethylenediaminetetraacetic acid (“EDTA”), sodium glycocholate, sodium glycodeeoxycholate, sodium lauryl suflate, sodium salicylate, sodium taurocholate, sodium taurodeoxycholate, as well as certain sulfoxides and glycosides, and mixtures thereof.

### I. 5-HT₃ Antagonists

A wide variety of 5-HT₃ antagonists may be suitable for use with the compositions and methods described herein. For example, the 5-HT₃ antagonist may be basic, acidic, or amphoteric in nature. In general, the 5-HT₃ antagonists described herein have an ionized form and an un-ionized form. In some variations, the 5-HT₃ antagonist is initially present in at least a partially ionized form. In other variations, the 5-HT₃ antagonist is initially present in an un-ionized form.

As described in more detail below, the buffer systems of the described compositions help facilitate absorption of the 5-HT₃ antagonists across the oral mucosa. For example, in some variations, the buffer systems described herein help convert substantially all of the 5-HT₃ antagonist from its ionized form to its un-ionized form. In other variations, the described buffer systems help ensure that a 5-HT₃ antagonist, initially in an un-ionized form, remains in an un-ionized form. As will be apparent by the description provided herein, the selection of a suitable 5-HT₃ antagonist is limited only by the capacity of the antagonist to be placed, or maintained, in an un-ionized form by the buffer systems described herein.

As used herein, the term 5-HT₃ antagonist includes all pharmaceutically acceptable forms of the 5-HT₃ antagonist being described. For example, the 5-HT₃ antagonist may be in a racemic or isomeric mixture, or may be a solid complex bound to an ion exchange resin or the like. In addition, the 5-HT₃ antagonist may be in a solvated form. The term 5-HT₃ antagonist is also intended to include all pharmaceutically acceptable derivatives and analogs of the described antagonist, as well as their mixtures. The ionized forms of the 5-HT₃ antagonists include their salt forms, for example, succinates, tartarates, hydrochlorides, salicylates, citrates, maleates, carbamates, sulfates, nitrates, benzoates, mixtures thereof, and the like.

A. Basic 5-HT₃ Antagonists

For basic 5-HT₃ antagonists, the relationship between pH and the conversion from an ionized to an un-ionized form is governed by the following formula: pH=pKa+Log₁₀ (Un-ionized concentration/Ionized concentration). When the pH is the same as the pKa, equimolar concentrations of the un-ionized form and ionized form exist. When the pH is one unit higher than the pKa, the ratio of the un-ionized form to the ionized form is 91:9. Similarly, when the pH is two units higher than the pKa, the ratio of un-ionized form to the ionized form is 100:1. As noted above, the un-ionized form is lipophilic and, therefore, more capable of passing through the mucous membranes than the ionized form, which is lipophobic in nature. Accordingly, increasing the pH of the saliva favors conversion of the ionized form into the un-ionized form for basic agents, and the final pH may be determined by making use of the above formula for any basic 5-HT₃ antagonist.

Any number of basic 5-HT₃ antagonists may be selected for use with the compositions and methods described herein. In general, the 5-HT₃ antagonists consist of three main components: (1) an aromatic structure; (2) a carbonyl-containing linking moiety; and (3) an out-of-plane basic nitrogen containing heterocyclic group. These groups have the specific spatial arrangement shown below.

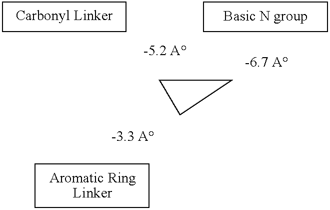

The 5-HT₃ antagonists are able retain their pharmacophore activity by either incorporating the carbonyl linker within the fused ring system, or by having the carbonyl group directly attached (as a spacer unit) to the aromatic ring and the basic nitrogen group. Those 5-HT₃ antagonists belonging to the former group may be represented by ondansetron. Illustrative examples of 5-HT₃ antagonists falling within this group are provided in Table 1. Those 5-HT₃ antagonists belonging to the latter group may be represented by granisetron. Illustrative examples of 5-HT₃ antagonists falling within this group are provided and in Table 2.

TABLE 1
The 5-HT₃ antagonists with carbonyl group incorporated within the
ring system.
Ar—R

| Drug | Ar | R |
|---|---|---|
| Ondan-setron | 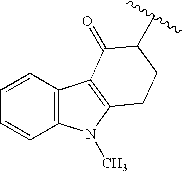 | 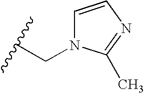 |
| Cilan-setron | 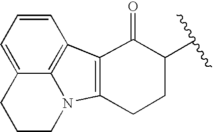 | 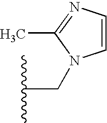 |
| Alo-setran | 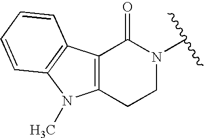 | 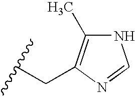 |
| Palono-setron | 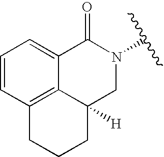 | 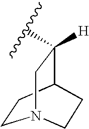 |

TABLE 2
The 5-HT₃ antagonists where the carbonyl group is attached to the
aromatic group and the nitrogen containing basic group as a spacer.

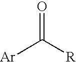

| Com- | | |
|---|---|---|
| pound | Ar | R |
| Grani-setron | 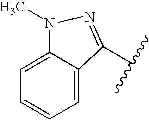 | 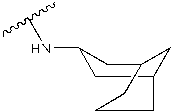 |
| Tropi-setron | 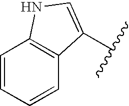 | 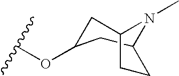 |
| Dola-setron | 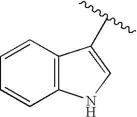 | 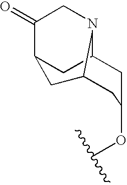 |
| Berne-setron | 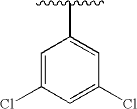 | 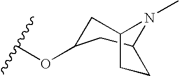 |
| Ramo-setron | 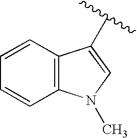 | 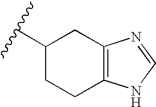 |
| Aza-setron | 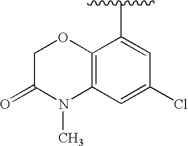 | 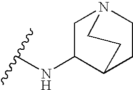 |
| Ita-setron | 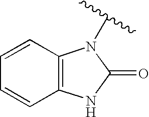 | 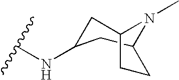 |
| Zaco-pride | 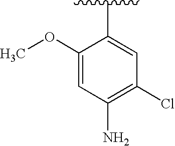 | 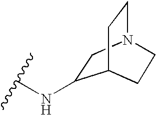 |

As can be seen by the examples provided in Table 1 and Table 2, the constant feature among the 5-HT₃ antagonists is the basic nitrogen group. The basic nitrogen group can be classified generally as imidazole (for example the N in ondansetron), or as a nitrogen-containing heterobicyclic derivative.

Using the above formula for basic 5-HT₃ antagonists, the overall lipophilicity and ionization activity of 5-HT₃ antagonists may be controlled and modulated by regulating the pH of the medium containing the 5-HT₃ antagonist relative to the pKa of the basic nitrogen group. The imidazole groups, when in conformational vicinity of an electron withdrawing carbonyl group, tend to have pKas in the region of about 7.4, and may be converted to their un-ionized, lipophilic form at a pH of about 9.4. In comparison, the 5-HT₃ antagonists containing nitrogen in a bicyclic ring, tend to have a pKa of about 8.8 and thus may be converted to their un-ionized, lipophilic form at a pH of about 10.8. As shown in Tables 1 and 2 above, examples of suitable 5-HT₃ antagonists include, but are not limited to ondansetron, palonosetron, tropisetron, lerisetron, alosetron, granisetron, dolasetron, bernesetron, ramosetron, azaseteron, itasetron, zacopride, cilansetron, and any other 5-HT₃ antagonist containing imidazole, oxazole, thiazole, pyrazole, 3-pyrroline or pyrrolidine in their structural formula.

B. Acidic 5-HT₃ Antagonists

Conversion of the ionized form to the un-ionized form for acidic 5-HT₃ antagonists is related to pH by the formula: pH=pKa+Log₁₀ (Ionized concentration/Un-ionized concentration). Accordingly, when the pH is the same as the pKa, equimolar concentrations of the un-ionized form and the ionized form exist at that pH. At one pH unit lower than the pKa, the ratio of the un-ionized form to the ionized form for an acidic agent is 91:9. Similarly, at two pH units lower than the pKa, the ratio of the un-ionized form to the ionized form for an acidic agent is about 100:1. Therefore, when the pH is two units lower than the pKa of an acidic 5-HT₃ antagonist, the acidic 5-HT₃ antagonist exists almost entirely in a lipophillic form. Accordingly, lowering the pH of the saliva favors conversion to the un-ionized form for acidic agents. Any number of acidic agents may be used with the described compositions and methods.

C. Amphoteric 5-HT₃ Antagonists

The 5-HT₃ antagonist may also be amphoteric. Amphoteric agents are those agents having both acidic and basic characteristics. In contrast to the basic and acidic 5-HT₃ antagonists, amphoteric 5-HT₃ antagonists are not modulated by the pKa of individual functional groups. Instead, they are modulated by the isoelectric point of the molecule. The isoelectric point is the pH at which the net charge on the molecule is zero.

### II. Buffer System

In general, the buffer systems of the compositions described herein are capable of changing the pH of saliva from an arbitrary initial pH to a predetermined final pH, independent of the arbitrary initial pH, and of sustaining the predetermined final pH for a period of time. In this way, the buffer systems help convert substantially all of the 5-HT₃ antagonist from its ionized form to its un-ionized form, or help ensure that a 5-HT₃ antagonist, initially in an un-ionized form, remains in an un-ionized form. The formulas provided above are used to determine the final pH at a given extent of ionization. The final pH is therefore dependent upon the nature of the 5-HT₃ antagonist (i.e., basic, acidic, or amphoteric), upon the pKa of the antagonist or its isoelectric point (for amphoteric agents), and upon the desired extent of conversion from the ionized form to the un-ionized form.

The final pH is typically determined when substantial conversion (e.g., greater than 50%, 80%, 90%, or 95%) to the un-ionized form is assumed. In this way the final pH favors substantially complete conversion to the un-ionized form when used with the described compositions in vivo. Once the final pH has been determined using the above formulas, buffering agents may then be selected to achieve that pH.

In some variations, the buffer system comprises a single buffering agent. In other variations, the buffer system comprises at least two different buffering agents. However, any number of buffering agents may be used as practicable, so long as the buffering system achieves the final pH, determined as described above. Similarly, a wide variety of different buffering agents may be used. In general, when a binary buffer system is used, the buffering agents comprise either a mixture of a weak acid and a salt of the weak acid, or a mixture of a first base and a second base, the second base being weaker than the first base. Illustrative examples of suitable buffering agents useful in either raising or lowering the pH of saliva include sodium carbonate, sodium bicarbonate, potassium carbonate, potassium bicarbonate, potassium citrate, mono basic potassium phosphate, magnesium oxide, magnesium carbonate, magnesium bicarbonate, alkaline starch, ascorbic acid, acetic acid, sodium acetate, and mixtures thereof.

Typically, the concentrations of the buffering agents are tailored such that the predetermined final pH of the saliva is achieved and sustained for a period of time, for example, at least 5 minutes, at least 10 minutes, or at least 20 minutes. This typically involves a trial and error type of procedure of adding various amounts of buffering agents and then measuring the final pH over time. In this way selection of an appropriate weight ratio for the given buffering agents may be easily determined in just a few trials. For binary systems, typically the buffering agents are in a weight ratio of from about 1:10 to 10:1. More typically, for binary systems, the buffering agents are in a weight ratio range of from about 1:2 to 2:1, of about 1:3 to 3:1, or of about 1:5 to 5:1. In some instances, the buffering agents are in a 1:1 ratio by weight for binary systems. Similar modifications may be made for tertiary or quaternary buffer systems, and the like.

In the case where acidic 5-HT₃ antagonists are used, the buffer system lowers the pH of the saliva. In these variations, the final pH of saliva is typically in the range of 1-6.9, and more typically 2-4. In the case where basic 5-HT₃ antagonists are used, the buffer system raises the pH of the saliva. In these variations, the final pH of saliva is typically in the range of 7.1-13, and more typically, 9-11. The final pH for amphoteric agents is dependent upon the isoelectric point of the molecule as described above, and therefore, the buffer system may either raise or lower the pH of saliva, depending upon the particular amphoteric agent chosen. In each instance, however, the final pH of saliva is typically of such a value that no damage is caused to the oral cavity or the mucous membranes. For example, a pH below 2 and a pH above 11.5 may be undesirable. Because of the limitations due to toxicity, it will be understood that in some situations, the extent of conversion to the un-ionized form may not reach 100%.

In addition to altering the pH of saliva, the buffer systems described herein may have the further advantage of altering the taste characteristics of the composition. This may be quite desirable when 5-HT₃ antagonists, which are very bitter, are chosen for transmucosal delivery. Typically, as the pH is lowered, the taste of the final composition becomes less bitter.

While the foregoing discussion has focused on the ability of the buffer system to alter salival pH in order to either favor substantially complete conversion to the un-ionized form, or to ensure that the un-ionized form does not convert back into the ionized form, it is conceivable that the buffer system may have subsidiary beneficial effects on the extent of absorption as well. For example, the buffer system may create a final salival pH that in turn effects the molecular configuration of the 5-HT₃ antagonist in a way in which absorption is increased. It is to be understood that these subsidiary beneficial effects of the buffer system are within the general scope of the buffer system and compositions herein described.

### III. Dosage Forms

While each individual possesses unique factors that may affect the rate and extent of absorption of the 5-HT₃ antagonists described herein, each of the described dosage forms offer advantages over the traditional dosage forms for oral administration. For example, each of the below dosage forms avoids hepatic first pass metabolism, degradation within the GI tract, and drug loss during absorption. Consequently, the amount of 5-HT₃ antagonist required per dose is less than would be required if formulated, for example, in a pill for oral administration.

Similarly, with each of the below dosage forms, the bioavailability of the 5-HT₃ antagonist is increased, and hence the time to onset of therapeutic activity is reduced. In addition, these dosage forms provide the advantage of allowing one who is already suffering from an upset stomach to avoid swallowing a pill, capsule, or tablet. In this way, the one suffering from the stomach upset avoids vomiting up the dosage form (which is problematic because the 5-HT₃ antagonist must first pass through the gastrointestinal system in order to produce a therapeutic effect). Instead, use of the dosage forms described herein allows the user to receive symptom relief (because the antagonist enters the bloodstream without first having to pass through the gastrointestinal system) without producing additional stomach discomfort.

A. Chewing Gum

In some variations, the final dosage form for the described compositions is in the form of a chewing gum. In general, the chewing gum compositions comprise at least one 5-HT₃ antagonist, a gum base, and a buffer system. In some variations, the chewing gum compositions further comprise a protecting agent as will be described in more detail below. Suitable 5-HT₃ antagonists and buffer systems were discussed in detail above. The percentage of 5-HT₃ antagonist in the chewing gum composition will vary depending upon the specific 5-HT₃ antagonist selected. Similarly, the total percentage of buffer system will vary depending upon the specific 5-HT₃ antagonist and the particular buffering agents selected.

Gum Base

In general, the gum base comprises a material selected from among the many water, and saliva, insoluble gum base materials known in the art. In some variations, the gum base comprises at least one hydrophobic polymer and at least one hydrophilic polymer. Illustrative examples of suitable polymers for gum bases include both natural and synthetic elastomers and rubbers, as well as mixtures thereof. Examples of suitable natural polymers include, but are not limited to, substances of plant origin like chicle, jelutong, gutta percha and crown gum. Examples of suitable synthetic elastomers include butadiene-styrene copolymers, isobutylene and isoprene copolymers (e.g., “butyl rubber”), polyethylene, polyisobutylene, polyvinylesters, such as polyvinyl acetate and polyvinyl acetate phthalate, and mixtures of any of the foregoing. In some variations, the gum base comprises a mixture of butyl rubber (a copolymer of isoprene and isobutylene), and polyisobutylene, and optionally, polyvinylacetate (preferably PVA having a MW of approximately 12,000).

Typically the gum base comprises from about 25% to about 75% of such polymers, and more typically, from about 30% to about 60%. Unless otherwise stated, all percentages provided herein are weight percentages, based on either the total weight of the gum base or of the final chewing gum composition, where noted.

The gum base may also include additional compounds, such as plasticizers (e.g., softeners or emulsifiers). These compounds may, for example, help reduce the viscosity of the gum base to a desirable consistency and improve its overall texture and bite. These compounds may also help to facilitate release of the active upon mastication. Non-limiting examples of these compounds include, lecithin, mono- and diglycerides, lanolin, stearic acid, sodium stearate, potassium stearate, glycerol triacetate, glycerol monostearate, glycerin, and mixtures thereof. The gum base typically comprises from about 0% to about 20% of plasticizer compounds, and more typically from about 5% to about 15%.

The gum base may further comprise waxes, such as beeswax and microcrystalline wax, or fats or oils, such as soybean and cottonseed oil. Typically, the gum base comprises from about 0% to about 25% of these waxes and oils, and more typically will comprise from about 15% to about 20%.

The gum base may further comprise one or more elastomeric solvents, for example, rosins and resins. Illustrative examples of such solvents include methyl, glycerol, and pentaerythritol esters of rosins or modified rosins, such as hydrogenated, dimerized or polymerized rosins or mixtures thereof (e.g., pentaerythritol ester of partially hydrogenated wood rosin, pentaerythritol ester of wood rosin, glycerol ester of wood rosin, glycerol ester of partially dimerized rosin, glycerol ester of polymerized rosin, glycerol ester of tall oil rosin, glycerol ester of wood rosin and partially hydrogenated wood rosin and partially hydrogenated methyl ester of rosin, such as polymers of alpha-pinene or beta-pinene, and terpene resins including polyterpene and mixtures thereof). Typically the gum base comprises from about 0% to about 75% of an elastomeric solvent, and more typically less than 10%.

The gum base may further comprise a filler material to enhance the chewability of the final chewing gum composition. Fillers that are substantially non-reactive with other components of the final chewing gum formulation are desirable. Examples of suitable fillers include calcium carbonate, magnesium silicate (talc), dicalcium phosphate, metallic mineral salts (e.g., alumina, aluminum hydroxide, and aluminum silicates), and mixtures thereof. Typically, the gum base comprises about 0% to about 30% of a filler, and more typically about 10% to about 20%.

The gum base may further comprise a preservative, such as butylated hydroxy toluene (“BHT”), and the like. Typically, the gum base comprises only trace amounts of a preservative, for example, less than about 0.1%.

The total chewing gum composition typically comprises from about 20% to about 90% of gum base, more typically less than about 70%, and most typically, from about 50% to about 60% of gum base. In certain instances the use of too much gum base may interfere with the release of the active ingredient, and additionally, may contribute to tackiness and poor mouth-feel of the final product. The use of a protecting agent, as described below may help to ameliorate this effect.

The chewing gum composition may further comprise at least one bulk sweetener to improve the palatability of the composition by masking any unpleasant tastes it may have. The sweetener may be incorporated into the gum base, but need not be. Examples of suitable sweeteners include compounds selected from the saccharide family, such as the mono-, di-, tri-, poly-, and oligosaccharides; sugars, such as sucrose, glucose (corn syrup), dextrose, invert sugar, fructose, lactose, maltodextrin, polydextrose; saccharin and its various salts, such as the sodium and calcium salts, cyclamic acid and its various salts; dipeptide sweeteners; chlorinated sugar derivatives such as sucralose, dihydrochalcone; and sugar alcohols such as sorbitol, sorbitol syrup, mannitol, xylitol, hexa-resorcinol and the like, including mixtures thereof. Hydrogenated starch hydrolysate, and the potassium, calcium and sodium salts of 3,6-dihydro-6-methyl-1-1,2,3-oxathiazin-4-one-2,2-dioxide may also be used. Of the foregoing, sorbitol, mannitol, xylitol, and lactose either alone or in combination, are most typically used. The chewing gum composition typically comprises from about 5% to about 75% of the sweetener, more typically from about 25% to about 40%, and most typically from about 30% to about 35%.

The chewing gum composition may further comprise one or more flavoring agents. The flavoring agent may be natural, synthetic, or a combination thereof. Examples of suitable flavoring agents include peppermint, spearmint, wintergreen, cinnamon, menthol, cherry, strawberry, watermelon, grape, banana, peach, pineapple, apricot, pear, raspberry, lemon, grapefruit, orange, plum, apple, fruit punch, passion fruit, chocolate (white, milk, dark), vanilla, caramel, coffee, hazelnut, mixtures thereof, and the like. Coloring agents (natural, artificial, or a combination thereof) may also be used. The coloring agents may also be used to color code the composition, for example, to indicate the type and dosage of the therapeutic agent therein. Suitable coloring agents include FD & C coloring agents, natural juice concentrates, pigments such as titanium oxide, silicon dioxide, and zinc oxide, and the like. The chewing gum composition typically comprises from about 0% to about 10% of the flavoring and coloring agents, either alone or in combination. More typically, the chewing gum composition comprises from about 0.1% to about 5% of the flavoring and coloring agents, and even more typically, from about 2% to about 3%.

The gum base need not be prepared from its individual components. The gum base may be purchased with the desired ingredients therein, and may or may not be modified. Several manufacturers produce gum bases, which may be suitable for use with the described chewing gum compositions. Examples of such suitable gum bases are the Pharmagum™ M, S, or C, sold by SPI Pharma Group in New Castle, Del. In general, the Pharmagum™ are a mixture of gum base, sweetener, plasticizer, and sugar. Literature on Pharmagum™ is readily available from its manufacturer, SPI Pharma Group, 321 Cherry Lane, New Castle, Del. 19720-2780.

In some variations, the chewing gum composition includes a therapeutic agent centerfill. A centerfill may be particularly suitable when immediate release of the 5-HT₃ antagonist is especially desirable. In addition, encapsulating the 5-HT₃ antagonist in a centerfill may help to mask any undesirable taste the 5-HT₃ antagonist may have.

In these variations, the gum base surrounds, at least in part, a centerfill. The centerfill comprises at least one 5-HT₃ antagonist, and may be a liquid or semi-liquid material. In some variations, the centerfill material may be low-fat or fat free. The centerfill may also contain one or more sweeteners, flavoring agents, coloring agents, and scenting agents as described herein. In some variations, the centerfill further includes a buffer system as described herein. In one variation, the centerfill comprises a combination of saccharide material, flavoring agent, a polyol, and an edible gel material.

Protecting Agent

The chewing gum composition may further comprise a protecting agent. The protecting agent coats at least part of the 5-HT₃ antagonist, typically upon the mixing of the two agents. The protecting agent may be mixed with the 5-HT₃ antagonist in a ratio of about 0.1 to about 100 by weight, more typically in a ratio of about 1 to about 50 and most typically in a ratio of about 1 to about 10.

The protecting agent reduces the adhesion between the 5-HT₃ antagonist and the gum base so that the 5-HT₃ antagonist may be more easily released from the gum base. In this way, delivery across the mucous membranes in about 5 to about 20 minutes of chewing, and desirably within about 10 minutes of chewing, is facilitated. A variety of different protecting agents may be used. Examples of suitable protecting agents include calcium stearate, glycerin monostearate, glyceryl behenate, glyceryl palmitostearate, hydrogenated castor oil, hydrogenated vegetable oil type I, light mineral oil, magnesium lauryl sulfate, magnesium stearate, mineral oil, poloxamer, polyethylene glycol, sodium benzoate, sodium chloride, sodium lauryl sulfate, stearic acid, cab-o-sil,® talc, zinc stearate, and mixtures thereof.

Methods of Making Chewing Gum Compositions

In some variations, the chewing gum composition is made from gum base granules. In these variations, the gum base takes the form of granules, with the 5-HT₃ antagonist interspersed among the granules. The gum base granules together with the 5-HT₃ antagonist are then compressed together to yield the final formulation.

In these variations, the chewing gum composition may be prepared using the procedures set forth in U.S. Pat. No. 4,405,647, which is hereby incorporated by reference in its entirety. According to the procedures set forth therein, the gum base material may be melted or softened using one or more of the softening agents, plasticizers and/or solvent and filler materials as described above. The sweeteners and flavoring agents are then mixed into the gum base by mixing the gum base material together with the water-soluble ingredients in a bed or blender within a gaseous medium at approximately 25° C. The resultant material is then continuously pulverized and chopped into smaller particles. To prevent adherence of the resultant particles to one another, filler or bulking material may be added, such as lubricants, glidants or any other tableting or compression aid, such as silica gel or calcium carbonate. Granules of any desired size and shape may be obtained when a standard mesh screen is selected to separate them.

The 5-HT₃ antagonist is then prepared to be mixed with the formed particulates such that the active is released within 20 minutes of chewing, and more desirably, within 5 to 15 minutes, and most desirably within 10 minutes. This can be done, for example, by mixing the 5-HT₃ antagonist with a protecting agent as described above. Once the 5-HT₃ antagonist is coated at least in part by the protecting agent, gum base is then continuously mixed into the 5-HT₃ antagonist/protecting agent mixture. During this mixing procedure, the 5-HT₃ antagonist/protecting agent (and gum base, if already added) mixture is typically always in an excess amount relative to the incoming, or newly added, gum base. In this way, the newly added gum base is diluted by the 5-HT₃ antagonist/protecting agent and gum base mixture.

Upon thorough mixing (using any suitable device), the materials are then compressed and compacted in a tablet press, or the like. In this way the 5-HT₃ antagonist is sandwiched in the voids between the compressed particulate gum granulate material and vice versa. The 5-HT₃ antagonist is thus made “external” to the gum base material itself. In one variation, the 5-HT₃ antagonist together with the particulates, heretofore described, are provided in a substantially non-liquid format. That is, the formulation of the invention according to this embodiment is preferably substantially 0% liquid.

In some variations, the gum base granules are further mixed together with the buffer system described herein. In other variations, the gum base granules are mixed with a single buffering agent, with at least one additional buffering agent being mixed with the 5-HT₃ antagonist. In still other variations, the gum base granules are mixed with half of the buffer system, with the other half being mixed with the 5-HT₃ antagonist.

The variations of the chewing gum compositions comprising a centerfill may be prepared using methods known in the confectionery and chewing gum industries. For example, U.S. Pat. No. 3,806,290, which is hereby incorporated by reference in its entirety, describes a method for forming a centerfill chewing gum by extruding a hollow-centered rope of chewing gum through an orifice having a pair of concentric conduits extending therethrough. A centerfill material is fed through the inner conduit to the hollow center upstream through a space between the inner and outer conduits. The centerfill rope of chewing gum is then passed to a sizing unit having a plurality of pairs of rollers for progressively decreasing a cross-sectional dimension of the gum rope. The plurality of pairs of rollers includes at least one vertical pair of rollers having vertically aligned axes of rotation and overlapping lower flange portions. A ramp structure is provided for guiding the gum rope above the roller flange portions upon entry of the gum rope between the vertical pair of rollers, to produce the final gum. Other methods of forming centerfill chewing gum known in the art may also be utilized.

The chewing gum compositions can have any desired shape, size, and texture. For example, the composition may have the shape of a stick, tab, gumball, and the like. Similarly, the gum may be any desirable color. For example the gum may be any shade of red, blue, green, orange, yellow, violet, indigo, and mixtures thereof, and may be color coded as described above. The gum can be individually wrapped or grouped together in pieces for packaging by methods well known in the art.

B. Slow-Dissolving Sublingual Tablets or Chewable Tablets

The compositions described herein may also be formulated as slow dissolving sublingual tablets or chewable tablets. In some variations, the compositions are formulated as slow-dissolving sublingual tablets, lozenges or candies. These types of dosage forms are held in the mouth, preferably below the tongue and are slowly dissolved by the user's saliva. Any type of lozenge or candy, having any number of desirable shapes and sizes may be used with the compositions described herein. A general discussion of lozenges and candies is provided in H. A. Lieberman, Pharmaceutical Dosage Forms, Volume 1: Tablets (1989), Marcel Dekker, Inc., New York, N.Y. at Medicated Confections, pages 75-418, which is hereby incorporated by reference in its entirety.

In general, these tablets comprises a 5-HT₃ antagonist and a buffer system. The 5-HT₃ antagonist and the buffer system are described in detail above. The formulation differs from that of the above described chewing gum compositions in that the water insoluble gum base is typically replaced by a fully or a partially water soluble natural or synthetic gum or binder. For example, in some variations, a suitable lozenge may be formed by replacing the Pharmagum™ described above with gum acacia or other appropriate gums or binders. The lozenges may optionally comprise diluents, disintegrants, flavoring agents, coloring agents, and scenting agents.

Typically the sublingual or chewable tablet base comprises from about 25% to about 80% of a fully or a partially water soluble synthetic or natural diluent, such as mannitol, sorbitol, dicalcium phosphate, calcium sulfate, lactose, cellulose, kaolin, mannitol, sodium chloride, or powdered sugar. Diluents such as mannitol, sorbitol, lactose, sucrose, inositol may impart properties to tablet that permit disintegration in mouth by chewing. Typically these diluents may be pre-processed to improve their flowability and taste. These methods include freeze drying, solid-solution preparation, lubricant dusting, and wet-granulation preparation with a suitable lubricant. Typically the diluents comprise about 50% to about 90% of the formulation and more typically about 60% to about 80%. Unless otherwise stated, all percentages provided herein are weight percentages, based on either the total weight of the 5-HT₃ antagonist or of the final sublingual or chewable tablet formulation, where noted.

The diluents can be freeze-dried using procedures set forth in Fundamentals of Freeze-Drying; Pharm. Biotechnol. 2002; volume 14 pages 281-360 and Lyophililization of Unit Dose Pharmaceutical Dosage Forms, Drug. Dev. Ind. Pharm. 2003; volume 29 pages 595-502, which are hereby incorporated by reference in their entirety. The solid-solution of diluents can be prepared by the procedures set forth in U.S. Pat. No. 6,264,987, which is hereby incorporated by reference in its entirety. Similarly, pre-processing using the lubricant dusting and wet granulation methods are set forth in “Remington—The Science and Practice of Pharmacy, 20^{th} Edition, Lippincott, Williams and Wilkins,” of which pages 865-868 are hereby incorporated by reference in their entirety.

The sublingual or chewable tablets may also include additional compounds, such as plasticizers (e.g., softeners or emulsifiers). These compounds may, for example, help reduce the viscosity of the salivary solution of the dissolved material to a desirable consistency and improve its overall texture and bite. These compounds may also help facilitate the release of the active upon chewing, in the case of chewable tablets. Non-limiting examples of these compounds include, lecithin, mono- and diglycerides, lanolin, stearic acid, sodium stearate, potassium stearate, glycerol triacetate, glycerol monostearate, glycerin, and mixtures thereof. The gum base typically comprises from about 0% to about 20% of plasticizer compounds, and more typically from about 5% to about 15%.

The sublingual or chewable tablet may also comprise binders, or granulators, to impart cohesive qualities to the powdered material. Non-limiting examples of these binders include starch, gelatin, sucrose, glucose, dextrose, molasses, lactose, acacia, sodium alginate, extract of Irish moss, panwar gum, ghatti gum, mucilage of isapol husks, carboxymethylcellulose, polyvinylpyrrolidone, Veegum,® larch arabogalactan, polyethylene glycol, ethyl cellulose, beeswax and microcrystalline wax, and fats or oils, such as soybean and cottonseed oil. Typically, the formulation comprises from about 0% to about 25% of these waxes and oils, and more typically comprises from about 15% to about 20%.

The sublingual or chewable tablet may further comprise one or more elastomeric solvents, for example, rosins and resins. Illustrative examples of such solvents include methyl, glycerol, and pentaerythritol esters of rosins or modified rosins, such as hydrogenated, dimerized or polymerized rosins or mixtures thereof (e.g., pentaerythritol ester of partially hydrogenated wood rosin, pentaerythritol ester of wood rosin, glycerol ester of wood rosin, glycerol ester of partially dimerized rosin, glycerol ester of polymerized rosin, glycerol ester of tall oil rosin, glycerol ester of wood rosin and partially hydrogenated wood rosin and partially hydrogenated methyl ester of rosin, such as polymers of alpha-pinene or beta-pinene, and terpene resins including polyterpene and mixtures thereof). Typically the sublingual or chewable tablet formulations comprises from about 0% to about 75% of an elastomeric solvent, and more typically less than 10%.

The sublingual or chewable tablet may also comprise lubricants to prevent adhesion of the tablet material to the surface of the dies and punches, and to reduce inter-particle friction. These lubricants may also facilitate ejection of the tablets from the die cavity and improve the rate of granulation flow during processing. Non-limiting examples of lubricants include talc, magnesium stearate, calcium stearate, stearic acid, hydrogenated vegetable oils, polyethylene glycol, and mixtures thereof. Typically, most of these lubricants, with the exception of talc, are employed at a concentration below 1%. Talc on the other hand may be used in concentrations as high as 10% and typically within a concentration range of 1 to 5%.

The sublingual or chewable tablets may further comprise at least one bulk sweetener to improve the palatability of the composition by masking any unpleasant tastes it may have. The sweetener may be incorporated into the diluent, but need not be. Examples of suitable sweeteners include compounds selected from the saccharide family, such as the mono-, di-, tri-, poly-, and oligosaccharides; sugars, such as sucrose, glucose (corn syrup), dextrose, invert sugar, fructose, maltodextrin, polydextrose; saccharin and its various salts, such as the sodium and calcium salts, cyclamic acid and its various salts; dipeptide sweeteners; chlorinated sugar derivatives such as sucralose, dihydrochalcone; and sugar alcohols such as sorbitol, sorbitol syrup, mannitol, xylitol, hexa-resorcinol and the like, including mixtures thereof. Hydrogenated starch hydrolysate, and the potassium, calcium and sodium salts of 3,6-dihydro-6-methyl-1-1,2,3-oxathiazin-4-one-2,2-dioxide may also be used. Of the foregoing, sorbitol, mannitol, xylitol, and lactose, either alone or in combination, are most typically used. The tablet composition typically comprises from about 5% to about 75% of the sweetener, more typically from about 25% to about 40%, and most typically from about 30% to about 35%.

The sublingual or chewable tablet composition may further comprise one or more flavoring agents. The flavoring agent may be natural, synthetic, or a combination thereof. Examples of suitable flavoring agents include peppermint, spearmint, wintergreen, cinnamon, menthol, cherry, strawberry, watermelon, grape, banana, peach, pineapple, apricot, pear, raspberry, lemon, grapefruit, orange, plum, apple, fruit punch, passion fruit, chocolate (white, milk, dark), vanilla, caramel, coffee, hazelnut, mixtures thereof, and the like. Coloring agents (natural, artificial, or a combination thereof) may also be used.

The coloring agents may also be used to color code the composition, for example, to indicate the type and dosage of the therapeutic agent therein. Suitable coloring agents include FD & C coloring agents, natural juice concentrates, pigments such as titanium oxide, silicon dioxide, and zinc oxide, and the like. The sublingual or chewable tablets typically comprise from about 0% to about 10% of the flavoring and coloring agents, either alone or in combination. More typically, they comprise from about 0.1% to about 5% of the flavoring and coloring agents, and even more typically, from about 2% to about 3%.

The diluents, binders and flavoring agents need not be prepared from its individual components. The diluents may be purchased with the desired ingredients therein, and may or may not be modified. Several manufacturers produce diluents, which may be suitable for use with the described tablet compositions. Examples of such suitable diluents are the Mannogem® and Sorbogem® sold by SPI Pharma Group in New Castle, Del. In general, the Mannogem® is a freeze-dried processed form of mannitol and Sorbogem® is a freeze-dried processed form of sorbitol. Literature on these diluents is readily available from their manufacturer, SPI Pharma Group, 321 Cherry Lane, New Castle, Del. 19720-2780.

In some variations, the sublingual or chewable tablet includes a centerfill. A centerfill may be particularly suitable when immediate release of the 5-HT₃ antagonist is especially desirable. In addition, encapsulating the 5-HT₃ antagonist in a centerfill may help mask any undesirable taste the 5-HT₃ antagonist may have.

The centerfill may comprise at least one 5-HT₃ antagonist, and may be a liquid or semi-liquid material. In some variations, the centerfill may be low-fat or fat free. The centerfill may also contain one or more sweeteners, flavoring agents, coloring agents, and scenting agents as described herein. In some variations, the centerfill further includes a buffer system as described herein. In one variation, the centerfill comprises a combination of saccharide material, flavoring agent, a polyol, and an edible gel material.

In another variation, the sublingual or chewable tablet is multilayered. In this way, the sublingual or chewable tablet may be designed to provide more than one pharmaceutically active agent. In this variation, for example, a 5-H T₃ antagonist may be combined with any other desirable pharmaceutical agent, or the 5-HT₃ antagonist may be combined with another 5-HT₃ antagonist. For example, in the case of a bi-layered tablet, the 5-HT₃ antagonist in the second layer may be the same or different from the agent incorporated in the first layer.

The first layer is typically, the sublingual or chewable portion of the tablet, and the second (and subsequent) layer is typically transposed over the original sublingual or the chewable layer. This type of formulation may be particularly suitable when immediate release of the 5-HT₃ antagonist is especially desirable, or when gastrointestinal absorption of a second therapeutic agent is desirable. Gastrointestinal absorption of a second agent may be desirable, for example, in order to mitigate co-morbid symptoms or to sustain the therapeutic benefit of the 5-HT₃ antagonist in the sublingual or the chewable portion of the tablet.

The second (and subsequent) layer(s) may also encapsulate the sublingual or chewable composition layer. The second layer may also be present as a layer lateral to the original sublingual or chewable composition layer. The second layer typically comprises at least one therapeutic agent, and may also comprise one or more sweeteners, flavoring agents, coloring agents, and scenting agents as described herein. In other variations, the second layer further includes a buffer system as described herein. It should also be noted that combinations of the 5-HT₃ antagonists with other desirable pharmaceutically active agents need not take the form of a discrete multilayered tablet. That is, the agents may simply be present throughout a single homogenous layer of the tablet. This type of formulation may also be used in the case where gastrointestinal absorption of at least one agent is desirable. In this scenario, the relative extent of ionization of the two agents will determine how they are to be absorbed. For example, those agents that are un-ionized will be absorbed through the oral mucosa, while those agents that are ionized will be swallowed for gastrointestinal absorption.

Protecting Agent

The sublingual or chewable tablets may further comprise a protecting agent. When employed, the protecting agent coats at least part of the 5-HT₃ antagonist, typically upon the mixing of the protective agent with the 5-HT₃ antagonist. The protecting agent may be mixed with the 5-HT₃ antagonist in a ratio of about 0.1 to about 100 by weight, more typically in a ratio of about 1 to about 50 and most typically in a ratio of about 1 to about 10.

The protecting agent reduces the adhesion between the 5-HT₃ antagonist and the diluents so that the 5-HT₃ antagonist may be more easily released from the formulation. In this way, the 5-HT₃ antagonist may be delivered across the mucous membranes in about 5 to about 20 minutes of chewing, and desirably within about 10 minutes. A variety of different protecting agents may be used. Examples of suitable protecting agents include calcium stearate, glycerin monostearate, glyceryl behenate, glyceryl palmitostearate, hydrogenated castor oil, hydrogenated vegetable oil type I, light mineral oil, magnesium lauryl sulfate, magnesium stearate, mineral oil, poloxamer, polyethylene glycol, sodium benzoate, sodium chloride, sodium lauryl sulfate, stearic acid, Cab-o-sil,® talc, zinc stearate, and mixtures thereof.

### Methods of Making Sublingual or Chewable Tablets

In some variations, the sublingual or chewable tablet composition is made from diluent granules. In these variations, the diluent takes the form of granules, with the 5-HT₃ antagonist interspersed among the granules. The diluent granules together with the 5-HT₃ antagonist are then compressed together to yield the final formulation.

In these variations, the sublingual or chewable tablets may be prepared using the procedures set forth in set forth in “Remington—The Science and Practice of Pharmacy, 20^{th} Edition, Lippincott, Williams and Wilkins,” as mentioned above. According to the procedures set forth therein, the diluent material may be milled or mixed. In addition, the diluent may be softened or lubricated using one or more of the softening agents, plasticizers and/or solvent and filler materials as described above. The diluent together with any of these softening agents, solvents, or fillers comprise the pre-mix. Sweeteners and flavoring agents, if desirable, are then added to the pre-mix. The pre-mix is then placed together with the water-soluble ingredients in a bed or blender within a gaseous medium or vacuum at approximately 25° C. The resultant material is then continuously pulverized and chopped into smaller particles. To prevent adherence of the resultant particles to one another, filler or bulking material may be added, such as lubricants, glidants or any other tableting or compression aid, such as silica gel or calcium carbonate. Granules of any desired size and shape may be obtained when a standard mesh screen is selected to separate them.

The 5-HT₃ antagonist is then prepared to be mixed with the formed particulates such that the 5-HT₃ antagonist is released within 20 minutes of sublingual placement or chewing, and more desirably, within 5 to 15 minutes, and most desirably within 5 to 10 minutes. This may be done, for example, by mixing the 5-HT₃ antagonist with a protecting agent as described above. Once the 5-HT₃ antagonist is coated at least in part by the protecting agent, the diluent is then continuously mixed into the 5-HT₃ antagonist/protecting agent mixture. During this mixing procedure, the 5-HT₃ antagonist/protecting agent (and diluents, if already added) mixture is typically always in an excess amount relative to the incoming, or newly added, diluent. In this way, the newly added diluent is diluted by the 5-HT₃ antagonist/protecting agent and diluent mixture.

Upon thorough mixing (using any suitable device), the materials are then compressed and compacted in a tablet press, or the like. In this way the 5-HT₃ antagonist is sandwiched in the voids between the compressed particulate diluent granulate material and vice versa. The 5-HT₃ antagonist is thus made “external” to the diluent material itself. In one variation, the 5-HT₃ antagonist together with the particulates, heretofore described, are provided in a substantially non-liquid format. That is, the formulation of the invention according to this embodiment is preferably substantially 0% liquid.

In some variations, the diluent granules are further mixed together with the buffer system described herein. In other variations, the gum base granules are mixed with a single buffering agent, with at least one additional buffering agent being mixed with the 5-HT₃ antagonist. In still other variations, the diluent granules are mixed with half of the buffer system, with the other half being mixed with the 5-HT₃ antagonist.

The sublingual and chewable tablets can have any desired shape, size, and texture. For example, the tablets may have the shape of a stick, tab, pellet, and the like. Similarly, the tablets may be any desirable color. For example the tablets may be any shade of red, blue, green, orange, yellow, violet, indigo, and mixtures thereof, and may be color coded as described above. The tablets can be individually wrapped or grouped together in pieces for packaging by methods well known in the art.

The sublingual and chewable tablets are primarily differentiated on the basis of location placement within the mouth, the time to tablet disappearance, and composition differences. Typically, the sublingual tablet is placed below the tongue and can be formulated to disappear (i.e., dissolve) immediately or gradually. Typically, the sublingual tablet is formulated to dissolve between about 1 to about 15 minutes, and more typically, between about 5 to about 10 minutes. This dissolution time would be reduced if the sublingual tablet were placed in any other location within the mouth (other then the sublingual space).

Unlike the sublingual tablet described just above, the chewable tablet is typically placed in the mouth ad hoc, often over the tongue. The chewable tablet is frequently moved around within the mouth and is sometimes even parked between the gums and the cheeks. The chewable tablet can also be formulated to dissolve immediately or gradually. Typically, the chewable tablet is formulated to dissolve between 1 to 15 minutes, and more typically, between 5 to 10 minutes. The dissolution time for the chewable tablets would be increased if it were placed in the sublingual space.

As noted above, the sublingual and chewable tablets are typically formulated to dissolve between 1 to 15 minutes, and more typically, between 5 to 10 minutes. However, while these time frames are amenable to maximum exposure of the drug (e.g., to the sublingual or buccal tissues), they are not always amenable to user compliance (i.e, users typically swallow too frequently and, therefore, hinder maximal transmucosal absorption). Consequently, it may be desirable to strike a balance between patient compliance and maximum exposure time of the drug in certain circumstances.

This may be accomplished in a number of ways. For example, the tablet size may be reduced without reducing the concentration of the buffer system (e.g., a reduction from a 700 mg to a 100 mg tablet, etc.). In addition, subtle changes to the tablet formulation may be used to reduce salivation. For example, chocolate may be used instead of spearmint as a flavoring agent in order to reduce salivation. Similarly, lactose may be used instead of mannitol or sorbitol (e.g., as a sweetner or diluent), to reduce salivation.

C. Quick-Dissolving Tablets

As indicated by their name, quick-dissolving tablets dissolve quickly after being placed within the mouth of a user. The tablet is dissolved by the user's saliva, without the need for chewing. This type of dosage form may be particularly desirable for pediatric and geriatric patients, since small children and aged individuals often have difficulty in chewing items.

In general, the quick-dissolving tablets comprise a 5-HT₃ antagonist and a buffer system. As with the chewing gum compositions and tablets described above, any number of flavoring agents or scenting agents may also be employed. Suitable 5-HT₃ antagonists and buffer systems are described above. The quick-dissolving tablet typically comprises a binder compound that is useful in keeping the tablet in a semi-solid state, and may be a solid, or a liquid. Similarly, the tablet may comprise a high-melting point fat or waxy material, or a disintegrant. Materials suitable as binders are discussed in detail above and may be used alone, or in combination, with the quick-dissolving tablets described here. The quick-dissolving tablets may be of any desirable shape, size, or color as described above.

### IV. Examples

The following examples are provided only to demonstrate various aspects of the compositions and methods described herein. It is to be understood that these examples are not comprehensive or exhaustive of the many variations of the compositions herein described. These examples are non-limiting, and for illustrative purposes only.

A. Ondansetron

Membrane Assay

The effect of pH adjustment on the extent of ionization, and hence, the extent to which a therapeutic agent will traverse the mucous membranes may be demonstrated by a membrane assay, see Kansy, M., Senner, F., Gubernator, K., 1998. “Physicochemical high throughput screening: parallel artificial membrane assay in the description of passive absorption processes.” J. Med. Chem., 41, 1007-1010; and Avdeef, “A. Physicochemical profiling (solubility, permeability, and charge state).” Curr. Topics Med. Chem. 2001, 1, 277-351. This assay uses a lipid-coated membrane to predict lipid mucosal membrane penetration.

The membrane apparatus consists of a dodecane membrane sandwiched between a donor and acceptor cell. The lipid-coated membrane is less porous then the mucous membranes of the oral cavity. Thus, the enhancement seen in the membrane assay is very likely to be magnified in vivo.

Membrane assays were performed using ondansetron HCl solutions at a pH of 5.4, 7.4 and 8.5. The alkaline pH values of 7.4 and 8.5 were adjusted using freshly prepared 0.01 M sodium bicarbonate-sodium carbonate buffer solution. The acidic pH of 5.4 was achieved using a 0.01 M acetate (sodium acetate & acetic acid) buffer solution. Permeation through the membrane was measured by determining the concentrations of ondansetron in the acceptor cell and is expressed as Pₑ (effective permeability in centimeters per second).

The results shown in Table 3 below demonstrate that the effective permeability of ondansetron HCl increases by more than 200% at a pH of 8.5 relative to the corresponding pH of 7.4 and 1000% relative to the pH of 5.4.

TABLE 3
Effective Permeability of Ondansetron HCl in Membrane Assay.

| pH | Pₑ (cm/s) |
|---|---|
| 5.4 | 0.32 |
| 7.4 | 1.30 |
| 8.5 | 3.25 |

Chewing Gum

The ondansetron may be formulated as a chewing gum composition as described above. In these variations, the unit dose, or serving for the chewing gum composition comprises about 0.1 to about 100 milligrams of ondansetron (as measured in its free base form), more desirably, from about 1 to about 50 milligrams, and most desirably from about 2 to about 25 milligrams. In some variations, it may be particularly desirable to include about 2-5 milligrams of ondansetron per serving, with perhaps 4 milligrams being especially desirable. Extra ondansetron, up to about 10-25% or so by weight may be added as “overage” or, the amount that may be expected to be “washed away” and not otherwise released or absorbed during mastication.

Given in weight percentages, the total amount of ondansetron (in whatever chosen form, measured as per its free base form) will typically be in the range of about 0.01% to about 10%, more typically from about 0.05% to about 2.0%, and most typically, from about 0.1% to about 1.0%. In some variations, 0.25% is particularly desirable. The foregoing percentages will vary depending upon the particular source of ondansetron, the amount of ondansetron desired in the final formulation, and on the desired release rate of the ondansetron.

The buffer system of the ondansetron chewing gum composition should result in a final salivary pH in excess of at least about 7.5, and even more desirably in the range from about 8 to about 10. A pH of at least about 9.5 is most desirable.

The ondansetron chewing gum containing the corresponding buffer system can be used for treatment of emesis, caused by a variety of clinical and pathological reasons, particularly the nausea and vomiting associated with cancer chemotherapy and radiotherapy, see, Green et al., Cancer Chemother. Pharmacol., 24:137-139 (1989). After introduction of a serving size piece of the gum composition into the mouth, the consumer will chew the gum as is normally done with any non-medicated type of chewing gum for about 20-30 minutes, but at approximately an average rate of about 10-45 chews per minute. The gum is then discarded.

A serving of the ondansetron chewing gum delivery system is typically designed to cause a loaded ondansetron concentration level in the bloodstream of at least about 10 to 300 nanograms of ondansetron per milliliter of plasma. For example, a 24 mg ondansetron chewing gum may be designed to produce a mean peak plasma concentration within the range of 150 to 300 nanograms of ondansetron per milliliter of plasma in 5 minutes to 2 hours. Similarly, an 8 mg dose may be designed to produce a mean peak plasma concentration within the range of 25 to 100 nanograms of ondansetron per milliliter of plasma in 5 minutes to 2 hours.

Pharmacokinetics

The pharmacokinetics of an 8 mg slow-dissolving sublingual ondansetron composition made by the methods described above was compared with the pharmacokinetics of an 8 mg Zofran® tablet, in six healthy male volunteers (i.e., the tablets were formulated to produce an 8 mg dose of ondansetron). The study was randomized and the drugs were administered in a 2-way crossover fashion after an overnight fast (10 hours) and an intermittent wash-out period of 4 days. The slow-dissolving ondansetron tablet was administered sublingually, and the volunteers were instructed to move the tablet under their tongue until the tablet dissolved (dissolution time about 4 to 8 minutes). The volunteers were also instructed to swallow saliva every minute until the tablet disappeared. Blood samples were drawn at 0, 4, 6, 8, 10, 13, 16, 20, 25, 30 40 and 50 minutes and at 1, 1.5, 2, 3, 4, 6, and 8 hours, respectively. Each blood sample was assayed for ondansetron concentration.

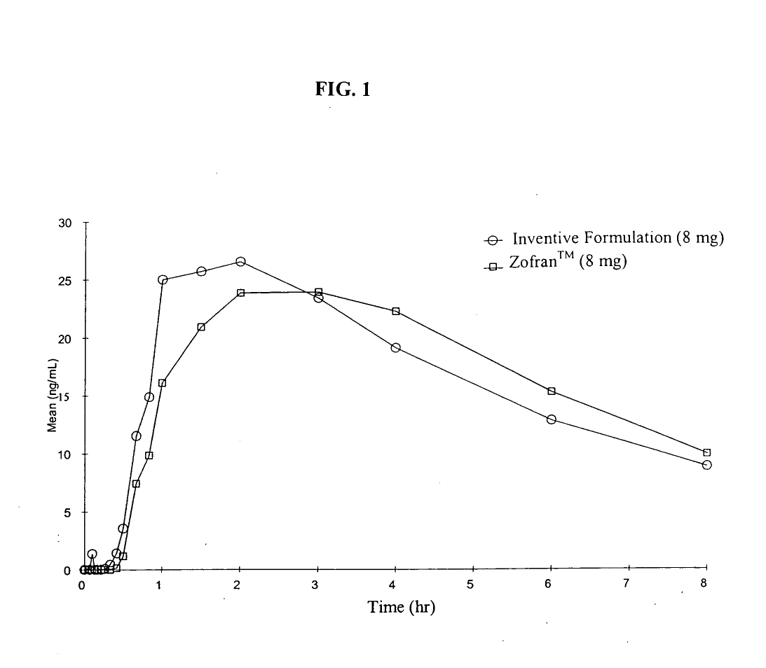

The results are shown in Table 4 below, and graphically represented in FIG. 1. Notably, while the maximum plasma concentration values (Cₘₐₓ) were similar for the two formulations (about 28 ng/ml), the time to achieve the maximum plasma concentration (Tₘₐₓ) was shorter for the slow-dissolving sublingual ondansetron formulation produced by the methods herein described. Further, the Cₘₐₓ/Tₘₐₓ for the slow-dissolving sublingual ondansetron formulation was about two-times the Cₘₐₓ/Tₘₐₓ value for the 8 mg Zofran® tablet. The 1-hour area under the curve (AUC) of the slow-dissolving sublingual ondansetron formulation, the shorter Tₘₐₓ, and a plasma profile typical of a rapid absorption, supports sublingual absorption.

TABLE 4
Individual and mean values of selected pharmacokinetic parameters of ondansetron
after an oral dose of an 8 mg Zofran ® tablet and an 8 mg slow-dissolving
sublingual ondansetron tablet of the present invention.

| | | Cₘₐₓ | | Tₘₐₓ | Cₘₐₓ/Tₘₐₓ |
|---|---|---|---|---|---|
| Treatment | Subject | (ng/ml) | AUClast | (min) | (ng/ml × min) |
| Slow-dissolving 8 mg | 1 | 35.67 | 8996.53 | 60 | 0.595 |
| sublingual ondansetron | 2 | 18.29 | 6381.98 | 120 | 0.152 |
| tablet | 3 | 22.75 | 7075.83 | 120 | 0.190 |
| | 4 | 31.59 | 8042.6 | 90 | 0.351 |
| | 5 | 36.73 | 10897.03 | 60 | 0.612 |
| | 6 | 23.73 | 6400.88 | 60 | 0.396 |
| | Mean | 28.127 | 7965.804 | 85 | 0.331 |
| | SD | 7.588 | 1755.558 | 29.49 | 0.257 |
| 8 mg Zofran ® Tablet | 1 | 35.09 | 9570.4 | 120 | 0.292 |
| | 2 | 20.93 | 6387.05 | 120 | 0.174 |
| | 3 | 24.67 | 6640.5 | 240 | 0.103 |
| | 4 | 29.32 | 8876.85 | 180 | 0.163 |
| | 5 | 33.73 | 10369.88 | 120 | 0.281 |
| | 6 | 24.45 | 6659.45 | 120 | 0.204 |
| | Mean | 28.032 | 8084.021 | 150 | 0.187 |
| | SD | 5.631 | 1735.275 | 50.2 | 0.112 |

The described methods and compositions provide a convenient, reliable, practical, and painless system for delivering 5-HT₃ antagonists across the oral mucosa. Notably, the described compositions are capable of rapidly delivering a 5-HT₃ antagonist so that a pharmacologically effective concentration of the 5-HT₃ antagonist enters the bloodstream within 20 minutes, 10 minutes, or even within 1-2 minutes after the 5-HT₃ antagonist is released from the carrier. Although the invention has been described with respect to certain variations, those of ordinary skill in the art may make modifications without departing from the scope and spirit of the invention.

What we claim is:

1. A composition for delivering a 5-HT₃ antagonist across the oral mucosa comprising:
at least one 5-HT₃ antagonist, wherein the 5-HT₃ antagonist is at least partly in an ionized form, the ionized form capable of being converted into an un-ionized form; and
a buffer system,
wherein the buffer system comprises at least two different buffering agents and is capable of changing the pH of saliva from an arbitrary initial pH to a predetermined final pH, independent of the arbitrary initial pH, and of sustaining the predetermined final pH for a period of time, and
wherein the predetermined final pH favors substantially complete conversion of the ionized form to the un-ionized form.

2. The composition of claim 1 wherein the predetermined final pH is within a range of from about 7.1 to about 11.5.

3. The composition of claim 2 wherein the predetermined final pH is within a range of from about 9 to about 11.

4. The composition of claim 1, wherein the composition is formulated as a lozenge, a chewing gum, or a dissolving tablet.

5. The composition of claim 4 formulated as a lozenge.

6. The composition of claim 4 formulated as a dissolving tablet.

7. The composition of claim 4 formulated as a chewing gum.

8. The composition of claim 1 further comprising a gum base.

9. The composition of claim 8 wherein the gum base comprises at least one hydrophobic polymer and at least one hydrophilic polymer.

10. The composition of claim 8, wherein the at least one hydrophilic polymer and the at least one hydrophobic polymer are independently selected from the group consisting of a natural polymer, a synthetic polymer, and mixtures thereof.

11. The composition of claim 10, wherein the at least one hydrophobic polymer is selected from the group consisting of a butadiene-styrene copolymer, butyl rubber, polyethylene, polyisobutylene, polyvinyl acetate phthalate, and mixtures thereof.

12. The composition of claim 11 wherein the hydrophobic polymer comprises a mixture of butyl rubber and polyisobutylene.

13. The composition of claim 1 wherein the predetermined final pH favors at least 80% conversion of the ionized form to the un-ionized form.

14. The composition of claim 13 wherein the 80% conversion occurs in 10 minutes or less.

15. The composition of claim 1 wherein the predetermined final pH favors at least 95% conversion of the ionized form to the un-ionized form.

16. The composition of claim 15 wherein the 95% conversion occurs in 10 minutes or less.

17. The composition of claim 1 wherein the predetermined final pH favors at least 99% conversion of the ionized form into the un-ionized form.

18. The composition of claim 17 wherein the 99% conversion occurs in 10 minutes or less.

19. The composition of claim 1 wherein the buffering agents are selected from the group consisting of a mixture of a weak acid and the salt of the weak acid, and a mixture of a first base and a second base, the second base being weaker than the first base.

20. The composition of claim 19 wherein the mixture of the first base and the second base are selected from the group consisting of sodium carbonate and sodium bicarbonate, potassium carbonate and potassium bicarbonate, and magnesium carbonate and magnesium bicarbonate.

21. The composition of claim 19 wherein the mixture of the weak acid and the salt of the weak acid is acetic acid and sodium acetate.

22. The composition of claim 20 wherein one buffering agent is sodium bicarbonate and one buffering agent is sodium carbonate.

23. The composition of claim 20 wherein one buffering agent is potassium bicarbonate and one buffering agent is potassium carbonate.

24. The composition of claim 19 wherein the buffering agents are in a weight ratio of from about 2:1 to 1:2.

25. The composition of claim 19 wherein the buffering agents are in a weight ratio of from about 3:1 to 1:3.

26. The composition of claim 19 wherein the buffering agents are in a weight ratio of from about 5:1 to 1:5.

27. The composition of claim 19 wherein the buffering agents are in a weight ratio of from about 10:1 to 1:10.

28. The composition of claim 19 wherein the buffering agents are in a 1:1 ratio by weight.

29. The composition of claim 1 wherein the period of time is at least 5 minutes.

30. The composition of claim 1 wherein the period of time is at least 10 minutes.

31. The composition of claim 1 wherein the period of time is at least 20 minutes.

32. The composition of claim 1 further comprising a penetration enhancer.

33. The composition of claim 1 wherein the 5-HT₃ antagonist is selected from the group consisting of ondansetron, palonosetron, tropisetron, lerisetron, alosetron, granisetron, dolasetron, bernesetron, ramosetron, azaseteron, itasetron, zacopride, and cilasentron.

34. The composition of claim 33 wherein the 5-HT₃ antagonist is ondansetron.

35. The composition of claim 34 wherein the ondansetron achieves a Cₘₐₓ/Tₘₐₓ within a range of about 0.15 ng/ml×min to about 0.6 ng/ml×min.

36. The composition of claim 35 wherein the ondansetron achieves a Cₘₐₓ/Tₘₐₓ within a range of about 0.4 ng/ml×min to about 0.6 ng/ml×min.

37. The composition of claim 34 wherein the membrane permeability of ondansetron is in the range of about 0.3 cm/s to about 3.25 cm/s.

38. A dissolving tablet for delivering a 5-HT₃ antagonist across the oral mucosa comprising:
a 5-HT₃ antagonist, wherein the 5-HT₃ antagonist is at least partly in an ionized form, the ionized form capable of being converted into an un-ionized form;
a protecting agent, wherein the protecting agent coats at least a portion of the 5-HT₃ antagonist; and
a buffer system,
wherein the buffer system comprises at least two different buffering agents and is capable of changing the pH of saliva from an arbitrary initial pH to a predetermined final pH, independent of the arbitrary initial pH, and of sustaining the predetermined final pH for a period of time, and
wherein the predetermined final pH favors substantially complete conversion of the ionized form to the un-ionized form.

39. The dissolving tablet of claim 38 further comprising a compound selected from the group consisting of a binder, a filler, a flavoring agent, a scenting agent, a coloring agent, a preservative, a plasticizer, a penetration enhancer, an elastomeric solvent, and mixtures thereof.

40. The dissolving tablet of claim 38 wherein the buffering agents are selected from the group consisting of a mixture of a weak acid and a salt of the weak acid, and a mixture of a first base and a second base, the second base being weaker than the first base.

41. The dissolving tablet of claim 40 wherein the mixture of the first base and the second base are selected from the group consisting of sodium carbonate and sodium bicarbonate, potassium carbonate and potassium bicarbonate, and magnesium carbonate and magnesium bicarbonate.

42. The dissolving tablet of claim 40 wherein the mixture of the weak acid and the salt of the weak acid is acetic acid and sodium acetate.

43. The dissolving tablet of claim 41 where one buffering agent is sodium bicarbonate and one buffering agent is sodium carbonate.

44. The dissolving tablet of claim 41 where one buffering agent is potassium bicarbonate and one buffering agent is potassium carbonate.

45. The dissolving tablet of claim 40 wherein the buffering agents are in a weight ratio of from about 2:1 to 1:2.

46. The dissolving tablet of claim 40 wherein the buffering agents are in a weight ratio of from about 3:1 to 1:3.

47. The dissolving tablet of claim 40 wherein the buffering agents are in a weight ratio of from about 5:1 to 1:5.

48. The dissolving tablet of claim 40 wherein the buffering agents are in a weight ratio of from about 10:1 to 1:10.

49. The dissolving tablet of claim 40 wherein the buffering agents are in a 1 to 1 ratio by weight.

50. The dissolving tablet of claim 38 wherein the 5-HT₃ antagonist is selected from the group consisting of ondansetron, palonosetron, tropisetron, lerisetron, alosetron, granisetron, dolasetron, bernesetron, ramosetron, azaseteron, itasetron, zacopride, and cilasentron.

51. The dissolving tablet of claim 50 wherein the 5-HT₃ antagonist is ondansetron.

52. A composition for delivering a 5-HT₃ antagonist across the oral mucosa comprising:
a 5-HT₃ antagonist, wherein the 5-HT₃ antagonist is at least partly in an un-ionized form, the un-ionized form capable of being converted into an ionized form at the normal salival pH; and
a buffer system,
wherein the buffer system comprises at least one buffering agent, and wherein the buffer system is capable of providing an adjusted salival pH such that the 5-HT₃ antagonist remains in its un-ionized form.

53. The composition of claim 52 wherein the buffer system is capable of maintaining the adjusted salival pH for a predetermined period of time.

54. The composition of claim 52 wherein the predetermined period of time is within the range of 5 to 10 minutes.

55. The composition of claim 52, wherein the composition is formulated as a lozenge, a chewing gum, or a dissolving tablet.

56. The composition of claim 55 formulated as a lozenge.

57. The composition of claim 55 formulated as a dissolving tablet.

58. The composition of claim 55 formulated as a chewing gum.

59. The composition of claim 52, wherein the buffer system comprises at least two different buffering agents.

60. The composition of claim 59 wherein the buffering agents are selected from the group consisting of a mixture of a weak acid and a salt of the weak acid, and a mixture of a first base and a second base, the second base being weaker than the first base.

61. A method for treating nausea comprising the steps of:
delivering a therapeutically effective amount of a 5-HT₃ antagonist across the oral mucosa.

62. The method of claim 61 wherein the step of delivering a therapeutically effective amount of a 5-HT₃ antagonist across the oral mucosa comprises the step of providing a composition comprising a 5-HT₃ antagonist and a buffer system
wherein the 5-HT₃ antagonist is at least partly in an un-ionized form, the un-ionized form capable of being converted into an ionized form at the normal salival pH, and
wherein the buffer system comprises at least one buffering agent, and wherein the buffer system is capable of providing an adjusted salival pH such that the 5-HT₃ antagonist remains in its un-ionized form.

63. The method of claim 61 wherein the step of delivering a therapeutically effective amount of a 5-HT₃ antagonist across the oral mucosa comprises the step of providing a composition comprising a 5-HT₃ antagonist, a protecting agent, and a buffer system
wherein the 5-HT₃ antagonist is at least partly in an ionized form, the ionized form capable of being converted into an un-ionized form,
wherein the protecting agent coats at least a portion of the 5-HT₃ antagonist, and
wherein the buffer system comprises at least two different buffering agents and is capable of changing the pH of saliva from an arbitrary initial pH to a predetermined final pH, independent of the arbitrary initial pH, and of sustaining the predetermined final pH for a period of time, and
wherein the predetermined final pH favors substantially complete conversion of the ionized form to the un-ionized form.

64. The method of claim 61 wherein the 5-HT₃ antagonist is selected from the group consisting of ondansetron, palonosetron, tropisetron, lerisetron, alosetron, granisetron, dolasetron, bernesetron, ramosetron, azaseteron, itasetron, zacopride, and cilasentron.

65. The method of claim 64 wherein the 5-HT₃ antagonist is ondansetron.
